(19) United States
(12) Patent Application Publication
(10) Pub. No.: US 2015/0018319 A1
(43) Pub. Date: Jan. 15, 2015

(54) TREATMENT OF SKIN DISEASE

(71) Applicant: Solutions International, LLC, Orem, UT (US)

(72) Inventors: Brian G. Larson, Alpine, UT (US); David Larson, Pleasant Grove, UT (US)

(73) Assignee: Solutions International, LLC, Orem, UT (US)

(21) Appl. No.: 14/195,123

(22) Filed: Mar. 3, 2014

Related U.S. Application Data
(63) Continuation-in-part of application No. 13/740,879, filed on Jan. 14, 2013.

(51) Int. Cl. A61K 31/616; A61K 31/60; A61K 9/00; A61K 47/26; A61K 47/10; A61K 31/327; A61K 47/02

(52) U.S. Cl. CPC A61K 31/616; A61K 31/327; A61K 31/60; A61K 47/02; A61K 47/26; A61K 47/10; A61K 9/0014
USPC 514/164; 514/714; 206/438

(57) ABSTRACT

The present invention is drawn to compositions, systems, and methods of treating skin disease. The composition includes a peroxygen, a transition metal or alloy thereof, and optionally, an alcohol and/or a drug. The composition can be packaged as part of a two-part system, and in one example, the drug is acetylsalicylic acid.

The present application is a continuation-in-part of U.S. patent application Ser. No. 13/740,879, filed on Jan. 14, 2013, which claims the benefit of U.S. Provisional Patent Application No. 61/586,485, filed on Jan. 13, 2012, each of which is incorporated herein by reference.

### BACKGROUND OF THE INVENTION

Acne and other skin disease and infection afflict many people, both on the face and elsewhere on the body. For example, acne is a skin condition that causes whiteheads, blackheads, and inflamed red growths in the form of papules, pustules, and cysts. Acne can occur when pores on the surface of the skin become blocked or clogged. The pores are present as an opening to a follicle, which contains a hair and an oil gland which act to lubricate the skin and help remove old skin cells. Thus, the oil that is present, along with dirt, debris, bacteria, cells, etc., often work together to cause the blockage. When the blockage breaks open, the material inside causes swelling and red bumps to form which often lead to firm and painful cysts.

There are also many other type of infections that have a negative impact on skin health, including inflammation, viral, bacterial, and fungal infections. Continued improvement in the treatment of skin afflicted with these and other types of diseases would be a benefit to those suffering from these types of conditions. Many drugs, including topically and orally administered drugs, have been used to treat skin infections and diseases to some success. However, it would be an advancement in the art to provide alternative systems and methods for enhancing skin health and treatment generally.

### SUMMARY OF THE INVENTION

In accordance with this, the present disclosure is drawn to compositions, systems, and methods of treating skin disease. In one embodiment, a composition suitable for treating skin disease can comprise water, from 0.5 wt % to 25 wt % glycerol and/or sorbitol, from 1 wt % to 30 wt % of a C₁ to C₂₄ alcohol, from 0.0001 wt % to 10.0 wt % of a peroxygen, from 0.0001 ppm to 50,000 ppm by weight of a transition metal or alloy thereof, and from 0.1 wt % to 8 wt % of a drug suitable for treating the skin disease. A related method of treating a skin disease can include applying this composition directly to a skin site afflicted with the skin disease.

In another example, a system suitable for treating skin disease can comprise a first container containing Part A and a second container containing Part B of a two-part solution. Part A can include a transition metal or alloy thereof, and Part B can include a peroxygen, an alcohol, and a drug. Upon combining Part A and Part B, a reacting formulation is formed that is effective for treating the skin disease. A related method of treating a skin disease can comprise obtaining this system, combining Part A and Part B to form a reacting formulation, and applying the reacting formulation to a skin site afflicted with the skin disease.

In another example, a composition suitable for treating skin disease can comprise water, an alcohol, a peroxide, a transition metal or alloy thereof, and acetylsalicylic acid. A related method of treating a skin disease can include applying this composition directly to a skin site afflicted with the skin disease.

In another embodiment, a system suitable for treating skin disease can comprise a first container containing Part A of a two-part solution where Part A includes a transition metal or alloy thereof. The system also includes a second container containing Part B of the two-part solution where Part B includes a peroxygen. The system also includes acetylsalicylic acid present in at least one of Part A, Part B, or a third formulation. Upon combining Part A and Part B, a reacting formulation is formed that, in combination with the acetylsalicylic acid, is effective for treating the skin disease. A related method includes obtaining this system, combining Part A and Part B to form a reacting formulation, and applying the reacting formulation along with the acetylsalicylic acid to a skin site afflicted with the skin disease. If the acetyl salicylic acid is already present in Part A or Part B, there may not be a third formulation present, as the acetylsalicylic acid is applied as part of Part A and/or Part B.

Additional features and advantages of the invention will be apparent from the detailed description that follows, which illustrates, by way of example, features of the invention.

### DETAILED DESCRIPTION

Reference will now be made to the exemplary embodiments, and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention. It is also to be understood that the terminology used herein is used for the purpose of describing particular embodiments only. The terms are not intended to be limiting unless specified as such.

It is noted that, as used in this specification and the appended claims, the singular forms “a,” “an,” and “the” include plural referents unless the content clearly dictates otherwise.

The terms “solution,” “composition” and “formulation” are also used throughout the specification to describe the compositions of the present disclosure. However, as these “solutions” can include colloidal transition metals, these compositions can also be described as dispersions or suspensions. As the continuous phase is typically a solution, and the transition metal can be present in ionic and/or colloidal form (and typically in small amounts and sizes), for convenience, these compositions will typically be referred to as “solutions,” “compositions” or “formulations” interchangeably. Further, sometimes a solution is referred to as a “resultant” solution or composition. This is to provide added clarity that the solution is a product of the mixing of a two (or more) part system. This being stated, the terms “solution” and “resultant solution” can be used interchangeably herein as is typically clear from the context of the discussion.

The term “reacting formulation” refers to compositions that are not at equilibrium, and in fact, often are actively reacting. For example, upon admixing a two-part formulation of the present disclosure, components form a reacting admixture that takes some time to come to equilibrium. During this reactive state after bringing the two-parts together, the resultant reacting formulation is more highly active for treating skin disease in accordance with embodiments of the present disclosure. In some examples, once equilibrium is reached, the formulation may not be as effective at treating skin disease as it was while actively reacting.

The term “peroxygen” refers to any compound containing a dioxygen (O—O) bond. Dioxygen bonds, particularly bivalent O—O bonds, are readily cleavable, thereby allowing compounds containing them to act as powerful oxidizers. Non-limiting examples of classes of peroxygen compounds include peracids, peracid salts, and peroxides, such as hydrogen peroxide or metal peroxides.

When referring to the term “alloy,” it is understood that individual colloidal or metallic particles can be in the form of composites of multiple metals, or alloys can also include co-dispersions of multiple metals as separate particles.

The term “two-part” when referring to the systems of the present disclosure is not limited to systems having only two parts. For example, the system can be a concentrate, and thus, is actually a three-part system, e.g., a first part including transition metal, a second part including a peroxygen, and a third part of a diluting solvent for diluting the first part, the second part, and/or the resultant solution. The third part could also include a drug, for example. Other ingredients can be present in the first part, the second part, the third, part, etc., such as alcohols, water, herbs, skin health additives, drug, etc. If diluting the first part, second part, or resultant solution, non-limiting examples of diluting solvents include water, alcohols, or combinations thereof. When the diluting solvent is an alcohol, it can, but need not be the same alcohol or mixture of alcohols which are present in the first or second “part” of the system. Thus, “two-part’ is specifically defined herein to mean, at least two-parts, unless the context dictates otherwise. Also, when referring to “Part A” or “Part B,” it is noted that the letter “A” or “B” is used merely for convenience, and does not infer which other co-ingredients may be present in a specific Part A or Part B formulation. Thus, “A” and “B” shall be interpreted to have no specific inference other than to identify an ingredient is from “this” part or the “other” part.

The term “container” refers to traditional containers such as tubes, dispensers, bottles, sprayers, etc. However, this term is to be viewed to be viewed more broadly to include fabrics (wipes), bandages, wrappings (foil, paper, etc.). Thus, anything capable of “containing” a fluid in accordance with embodiments of the present disclosure can be considered a container.

The term “drug” refers to any bioactive agent or agents which can be used to effectively treat skin disease or other skin infection or affliction, e.g., inflammatory, viral, fungal, bacterial, etc. Often, a single drug or active agent can be effective in treating multiple disease or infection types, or combination of multiple drugs can be used to treat a single or multiple infection types. Thus, no particular drug or combination thereof is to be associated specifically with a specific skin disease. A broad list of drugs is included herein, but any drug effective for treating skin disease can be used. In one specific example, the drug can be acetylsalicylic acid.

The term “subject” refers to any animal. In particular, subjects can be mammals, and more particularly humans.

The term “skin” includes human skin, nail, and mucosal surfaces that can suffer from various forms of disease and infection and are usually at least partially exposed to the environment, such as skin, nails, lips, and mucosa.

The term “skin disease” refers to any of a number of skin ailments and infections that afflict the skin surface or deeper skin tissue, including inflammatory skin disease (e.g., acne, eczema, dermatitis, poison ivy, psoriasis, pyoderma gangrenosum, rosacea, hives, inflamed burns, etc.); bacterial skin infection (e.g., impetigo, folliculitis, furunculosis, carbunculosis, eethyma, erysipelas, cellulitis, necrotizing fasciitis, etc.); fungal and yeast infection (e.g., dermatophytosis, candidiasis, tinea, athlete's foot, nail fungal infection, diaper rash, etc.); viral infection (e.g., herpes simplex, herpes zoster, cold sores, warts, molluscum contagiosum, etc.); and infection caused by small macro organisms such as mites (e.g., face mites such as demodex folliculorum, Demodex brevis, Demodex canis, etc.), insects, animals (bites), etc.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight ratio range of about 1 wt % to about 20 wt % should be interpreted to include not only the explicitly recited limits of 1 wt % and about 20 wt %, but also to include individual weights such as 2 wt %, 11 wt %, 14 wt %, and sub-ranges such as 10 wt % to 20 wt %, 5 wt % to 15 wt %, etc.

It is noted that when a range or value is given with respect to weight percent (wt %), the weight percent that is referred to is that in the resultant composition or formulation after the two-part system is brought together unless clearly stated otherwise. Thus, if it is stated that a drug is present in a formulation at from 3 wt % to 8 wt %, that indicates that the final composition that is applied the skin has drug present within that weight ratio range. This is primarily applicable to the two-part embodiments described herein, as one-part systems would not create any confusion as to the applicable weight ratio range. Thus, it is understood that the initial drug or other ingredient present in one of the two-parts may be greater than that in the resultant formulation, and may be outside of the range described in the composition that will ultimately be applied to the skin. Alternatively, in some instances, a weight percentage will be given and clearly labeled as being a weight percentage of one specific part of a two-part system (Part A or Part B), e.g., see certain examples. In those instances, the weight percentages shall be as indicated. Thus, in these “two-part” embodiments, it is notable that the concentrations of each ingredient can be described in the context of concentration in the first or second composition (when indicated by the context of the description), or the resultant solution or composition (as a default). The concentration of a compound in the first or second liquid composition will usually be lower in the resultant composition or solution than in the first or second liquid composition, as the amount typically gets diluted by the other part of the system. That being stated, this is not always the case, depending on the ingredients in the other portion of the two-part system. For example, if an ingredient is generated by a reaction, the amount may actually increase when the two-part system is combined to form the resultant composition, e.g., peracid and peroxide chemistry.

With this in mind, the present disclosure provides compositions, systems, and methods for treating and/or preventing skin disease of various types. In one embodiment, a composition suitable for treating skin disease can comprise water, from 0.5 wt % to 25 wt % glycerol and/or sorbitol, from 1 wt % to 30 wt % of a C₁ to C₂₄ alcohol, from 0.0001 wt % to 10.0 wt % of a peroxygen, from 0.0001 ppm to 50,000 ppm by weight of a transition metal or alloy thereof, and from 0.1 wt % to 8 wt % of a drug suitable for treating the skin disease. A related method of treating a skin disease can include applying this composition directly to a skin site afflicted with the skin disease.

In another example, a system suitable for treating skin disease can comprise a first container containing Part A and a second container containing Part B of a two-part solution. Part A can include a transition metal or alloy thereof, and Part B can include a peroxygen, an alcohol, and a drug. Upon combining Part A and Part B, a reacting formulation is formed that is effective for treating the skin disease. A related method of treating a skin disease can comprise obtaining this system, combining Part A and Part B to form a reacting formulation, and applying the reacting formulation to a skin site afflicted with the skin disease.

In another example, a composition suitable for treating skin disease can comprise water, an alcohol, a peroxide, a transition metal or alloy thereof, and acetylsalicylic acid. A related method of treating a skin disease can include applying this composition directly to a skin site afflicted with the skin disease.

In another embodiment, a system suitable for treating skin disease can comprise a first container containing Part A of a two-part solution where Part A includes a transition metal or alloy thereof. The system also includes a second container containing Part B of the two-part solution where Part B includes a peroxygen. The system also includes acetylsalicylic acid present in at least one of Part A, Part B, or a third formulation. Upon combining Part A and Part B, a reacting formulation is formed that, in combination with the acetylsalicylic acid, is effective for treating the skin disease. A related method includes obtaining this system, combining Part A and Part B to form a reacting formulation, and applying the reacting formulation along with the acetylsalicylic acid to a skin site afflicted with the skin disease.

In each of the various embodiments herein, whether discussing the compositions, systems, or methods, there may be some common features of each of these embodiments that further characterize options in accordance with principles discussed herein. Thus, discussions of the compositions, systems, or methods alone are also applicable to the other embodiments not specifically mentioned.

Though not required, typically, the therapeutic compositions of the present disclosure can be stored as a two-part system, and brought together prior to use, such as immediately prior to use, e.g., within 60 seconds, within 5 minutes of admixture, within 1 hour of admixture, within 24 hours of admixture, within 1 week of admixture. Longer periods of time may lead to the active ingredients becoming less effective, though to the extent that they remain effective for treating skin disease, any amount of time may be appropriate as long as the ingredients are effective for their intended use of treating skin disease. For example, a two-part system can be formulated to split up the ingredients between the two (or more) ingredients in any manner that preserves the activity of the ingredients for use when brought together. In accordance with this, the silver and the peroxygen are typically kept in separate containers to prevent premature activation of these ingredients prior to use. Other ingredients can typically be in either or both parts (or a third part).

As mentioned previously, in one example, the compositions of the present disclosure can comprise an aqueous vehicle including water, from 0.5 wt % to 25 wt % glycerol and/or sorbitol, from 1 wt % to 30 wt % of a C₁ to C₂₄ alcohol, from 0.0001 wt % to 10.0 wt % of a peroxygen, from 0.0001 ppm to 50,000 ppm by weight of a transition metal or alloy thereof, and from 0.1 wt % to 8 wt % of a drug suitable for treating the skin disease. Other ranges will likewise be provided hereinafter. It is thus noted that the lower end of the range of the peroxygen in the administered aqueous composition can be modified to 0.05 wt % or 0.1 wt %, and/or the upper end of the range can be modified to 5 wt %, 3 wt %, or 1.5 wt % in accordance with specific embodiments of the present disclosure. Further, the concentration of the metal content, including ionic and/or colloidal content, can be modified to 10 ppm, 1 ppm, 0.1 ppm, 0.01, or 0.001 by weight at the lower end of the range, and/or to 10,000 ppm, 5,000 ppm, or 1,500 ppm by weight at the upper end of the range. It is also noted that alcohol content generally (e.g., glycerol, sorbitol, aliphatic alcohol, or other formulations that include these or other alcohols) can be present at from 0.0001 wt % to 95 wt %. This being stated, the lower end of the range of the alcohol can be modified to 0.05 wt % or 0.1 wt %, and the upper end of the range can be modified to 40 wt %, 30 wt %, 20 wt % or 10 wt % in accordance with specific embodiments of the present disclosure. Furthermore, the drug can be present ranging from 0.1 wt % to 20 wt %, without limitation. The lower end of the range of the drug can be modified to 0.5 wt % or 1 wt %, and the upper end of the range can be modified to 10 wt %, 5 wt %, or 2 wt % in accordance with specific embodiments of the present disclosure, regardless of the drug selected. As these ranges are merely exemplary, one skilled in the art could modify these ranges for a particular application, considering such things as the type of alcohol (polyhydric, mixtures, etc.); the type of peroxygen (peroxide, peracid, combination of peroxide/peracid, etc.); the type of metal (ionic, colloidal, alloy, etc.), the type of drug, and the particular skin disease being treated. Further, it is noted that any combination of these upper and lower limits for each of the ingredients are expressly included herein.

Though specific ingredients are described herein in detail, it is noted that there will also typically be an aqueous vehicle that includes water and optionally other ingredients, such as organic co-solvents, surfactants, and the like, so long as the additional ingredients are compatible with the intended compositions, systems, and methods of treatment.

In one specific example, the composition of the present disclosure can be prepared by admixing at least two-parts together in accordance with a preliminary step of admixing a first liquid composition and a second liquid composition to form the composition suitable for treating skin disease. The first liquid composition (or Part A) can comprise the transition metal or alloy thereof, and the second liquid composition (or Part B) can comprise the peroxygen. Drug, alcohol, and other ingredients can be in one and/or both of these compositions, and/or can be in a third liquid composition. Alternatively, the first liquid composition (Part A) can comprise the peroxygen, and the second liquid composition (Part B) can comprise the transition metal or alloy thereof. Thus, it is not significant what ingredients are in Part A and what ingredients are in Part B, provided the parts that are reactive or interactive with one another are kept separate, e.g., colloidal metal separated from the peroxygen in some formulations, or the drug separated from any ingredient that would adversely impact its effectiveness. As mentioned, in some instances, it may be beneficial that the two-part system actually include three-parts if there are three ingredients that should be kept separate until just prior to use. To provide one example, the system may include a first liquid composition (Part A) including the transition metal or alloy thereof (e.g., silver), and a second liquid composition (Part B) including a peroxygen (e.g., hydrogen peroxide), alcohol (e.g., glycerol, sorbitol, lower alky (C₁-C₂₄) alcohol, etc.), and drug (e.g., salicylic acid or acetylsalicylic acid).

When a two-part solution is brought together, reactions occur that can also reduce or increase relative concentrations of given ingredients, e.g., in the case of peracid/peroxide compositions, the peroxide component of the peracid is rapidly converted into water and oxygen within minutes of activation, and ceases to exist in some cases. Additionally, such two-part embodiments can sometimes provide effective activation for a period of weeks, e.g., up to 60 days after activation, or more, depending on the specific composition. Furthermore, whether preparing a two-part system or a single solution, these compositions can be prepared so that they are non-corrosive or non-toxic, and emit no emissions into the environment. Furthermore, these solutions can be prepared so that they pose no health or safety issues, since all of the ingredients (except for the drug in some instances) are essentially food grade after activation. For example, in the case of some two-part systems of peracids and peroxides, e.g., peroxyacetic acid and hydrogen peroxide, after activation by bringing the two-parts together, the dramatically altered chemical form of the peracid post-activation is no longer corrosive to the skin, exhibits no oral or inhalation toxicities, no dermal toxicities, and only mild irritation when sprayed directly into the eyes (no permanent damage to the eyes). If the desire is to treat the eye with the formulations of the present disclosure, safe formulations for eye application can also be prepared.

Turning to the compositional components more specifically, in embodiments where a drug used, examples of such drugs that are usable for one or a variety of skin diseases or conditions in accordance with the present disclosure include, without limitation, benzoyl peroxide, salicylic acid, acetylsalicylic acid, sulfur, resorcinol, resorcinol monoacetate, amorolfine, butenafine, naftifine, terbinafine, fluconazole, itraconazole, ketoconazole, posaconazole, ravuconazole, voriconazole, clotrimazole, butoconazole, econazole, miconazole, oxiconazole, sulconazole, terconazole, tioconazole, caspofungin, micafungin, anidulafingin, amphotericin B, AmB, nystatin, pimaricin, griseofulvin, ciclopirox olamine, haloprogin, tolnaftate, undecylenate, acyclovir, penciclovir, famciclovir, valacyclovir, trifluridine, idoxuridine, cidofovir, gancyclovir, podofilox, podophyllotoxin, ribavirin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, rimantadine, zanamivir, erythromycin, clindamycin, tetracycline, bacitracin, neomycin, mupirocin, polymyxin B, quinolones, imiquimod, or combinations thereof. Other drugs that have a therapeutic effect with respect to certain types of skin disease can also be used and determined by one skilled in the art after considering the present disclosure with routine experimentation. Considerations for use would include activity with respect to a desired treatment of a skin disease, and compatibility with the other ingredients in the composition or during storage of at least one part of a two-part system.

In certain examples, there are certain specific drugs that can be used very effectively, with low side effects, on a variety of skin conditions, including but not limited to acne, warts, cold sores, eczema, psoriasis, athlete's foot, diaper rash, burns, etc., with acceptable results. These active agents or drugs include, without limitation, benzoyl peroxide (e.g., 2.5 wt % to 10 wt %), salicylic acid (e.g., 0.5 wt % to 2 wt %), acetylsalicylic acid (e.g., 0.5 wt % to 3 wt %), sulfur (e.g., 3 wt % to 10 wt %), resorcinol (e.g., 1 wt % to 3 wt %), and resorcinol monoacetate (e.g., 2 wt % to 4 wt %). These weight ratios are given by way of examples, and can be used outside of these ranges as described otherwise herein. Furthermore, a combination of these active ingredients can also be used, such as about 2 wt % resorcinol combined with 3 wt % to 8 wt % sulfur, or about 3 wt % resorcinol monoacetate combined with 3 wt % to 8 wt % sulfur. Other combinations can also be effective. It is noted that these weight percentages given are provided for general guidance only, and can be expanded in accordance with examples of the present disclosure.

If an alcohol is present in the composition, or in one or both of Part A and Part B of the two-part system, in one example, the alcohol can be present at from about 0.0001 wt % to 95 wt %, with the upper end and lower end of the range being modifiable as described previously. Examples of alcohols that can be used include, but are limited to, aliphatic alcohols and other carbon-containing alcohols, having from 1 to 24 carbons (C₁-C₂₄ alcohol). It is to be noted that “C₁-C₂₄ alcohol” does not necessarily imply only straight chain saturated aliphatic alcohols, as other carbon-containing alcohols can also be used within this definition, including branched aliphatic alcohols, alicyclic alcohols, aromatic alcohols, unsaturated alcohols, as well as substituted aliphatic, alicyclic, aromatic, and unsaturated alcohols, etc. In one embodiment, the aliphatic alcohols can be C₁ to C₅ alcohols including methanol, ethanol, propanol and isopropanol, butanols, and pentanols, due to their availability and lower boiling points. This being stated, polyhydric alcohols can also be used effectively in enhancing the potency of the solution of the present disclosure, as well as provide some degree of added stabilization. Examples of polyhydric alcohols which can be used in the present disclosure include but are not limited to ethylene glycol (ethane-1,2-diol), glycerin (or glycerol, propane-1,2,3-triol), sorbitol, and propane-1,2-diol. Other non-aliphatic alcohols may also be used including but not limited to phenols and substituted phenols, erucyl alcohol, ricinolyl alcohol, arachidyl alcohol, capryl alcohol, capric alcohol, yl alcohol, lauryl alcohol (1-dodecanol), myristyl alcohol (1-tetradecanol), cetyl (or palmityl) alcohol (1-hexadecanol), stearyl alcohol (1-octadecanol), isostearyl alcohol, oleyl alcohol (cis-9-octadecen-1-ol), palmitoleyl alcohol, linoleyl alcohol (9Z,12Z-octadecadien-1-ol), elaidyl alcohol (9E-octadecen-1-ol), elaidolinoleyl alcohol (9E, 12E-octadecadien-1-ol), linolenyl alcohol (9Z,12Z,15Z-octadecatrien-1-ol), elaidolinolenyl alcohol (9E,12E,15-E-octadecatrien-1-ol), combinations thereof, and the like.

In some embodiments, for practical considerations, methanol, ethanol, propanols, butanols, pentanols, and denatured alcohols (mixtures of ethanol and smaller amounts of methanol and other possible minor amounts of organics) can often be used because of their availability and cost. Glycerol or sorbitol can also be used in some embodiments. Since the desire is typically to provide a highly skin safe composition, alcohols can be selected that satisfy this desire. When considering the amount of alcohol to use, one skilled in the art can stay within the above-described ranges, or modify these ranges for a particular application, considering such things as whether alcohol selected for use is polyhydric, whether the alcohol is food grade, mixtures of alcohols, etc.

Regarding the transition metal or alloy, a concentration in the range of 0.0001 ppm to 50,000 ppm by weight can be used and/or modified as described previously. The metal can be in ionic form (e.g., disassociate metal salt, metal ions from elemental metal, etc.) and/or in colloidal form. In one specific embodiment, the transition metal can be in a sub-micron form (i.e. dispersion of less than 1 μm metal colloidal particles). However, larger colloidal transition metal particles can also be used in certain applications. Typical transition metals that are desirable for use include Group VI to Group XI transition metals, and more typically, can include Group X to Group XI transition metals. Alloys including at least one metal from the Group VI to Group XI metals can also be used. It is recognized that any of these metals will typically be oxidized to the corresponding cation in the presence of a peroxygen. However, with colloidal metals, typically, the surface is usually more susceptible to such oxidation. Further, when colloidal metals are dispersed in a colloidal solution, there is often an amount of the metal in ionic or salt form that is also present in the suspension solution. For example, colloidal silver may include a certain percentage of silver salt or ionic silver in solution, e.g., 10 wt % to 90 wt % of metal content can be ionic based on the total metal content. This being stated, certain metals for use in accordance with embodiments of the present disclosure are ruthenium, rhodium, osmium, iridium, palladium, platinum, copper, gold, silver, manganese, zinc, alloys thereof, and mixtures thereof. As mentioned, the transition metal can be colloidal or ionic. Colloidal metal can be elemental metal (or alloys of elemental metals), or can be in an insoluble salt form, such as zinc oxide or the like. For skin application, silver and zinc work well together, but metal choice can be dependent to some degree on the application, the levels of infection to be treated, etc.

It is also noted that any of these embodiments can often also benefit from the use of alloys. For example, certain combinations of metals in an alloy may provide benefits that are related more to other consideration, such as solution stability, substrate to be cleaned, etc. Examples of transition metal alloys for use in the present disclosure include but are not limited to copper-silver alloys, silver-manganese alloys, chromium-silver alloys, gold-silver alloys, magnesium-silver alloys, zinc-silver alloys, and the like.

Exemplary colloidal silvers that can be used in the first liquid composition include those sold by Solutions IE, Inc. under the trade names CS Plus and CS Ultra. Other colloidal silver products that can be used as the silver source include ASAP, Sovereign Silver, Silver Max, and the like. In one embodiment, the colloidal particles used in the present disclosure can have a particle size range of from 0.001 μm to 1.0 μm. In another embodiment, the colloidal transition metal particles can have a size range of from 0.030 μm to 0.5 μm. In still another embodiment the average particle size is 0.35 μm to 0.45 μm. If used in ionic form, silver salts can include but are not limited to silver nitrate, silver acetate, silver citrate, silver oxide, and/or silver carbonate. Though many colloidal silver solutions or ionic silver solutions that are functional for use in the formulations of the present disclosure can be used, in one embodiment, it can be desirable to use RO water as the suspension medium for the colloidal and/or ionic silver that is mixed with the other ingredients. In a more detailed aspect, the RO water can also be distilled, resulting in 18-20 MO water, though this is not required. Exemplary colloidal zinc that can be used includes that available from Purest Colloid, Inc. sold under the trade name MesoZinc, colloidal zinc available from Quality Colloids, Inc., and Zn1100 available from Solutions IE. Other sources of colloidal silver, colloidal zinc, and other colloidal or ionic metals are also generally available.

The peroxygen can be present in the compositions of the present disclosure at from 0.0001 wt % to 10 wt %, with the upper end of the range being modifiable as described previously herein. The peroxygen can be a single compound or a combination of multiple peroxygen compounds or peroxygen forming compounds. In one embodiment, the peroxygen can be any aliphatic or aromatic peracid (or peroxyacid) that is functional for treatment purposes in accordance with embodiments of the present disclosure. While any functional peroxyacid can be used, peroxyacids containing from 1 to 7 carbons are the most practical for use. These peroxyacids can include, but not be limited to, peroxyformic acid, peroxyacetic acid, peroxyoxalic acid, peroxypropanoic acid, perlactic acid, peroxybutanoic acid, peroxypentanoic acid, peroxyhexanoic acid, peroxyadipic acid, peroxycitric, and/or peroxybenzoic acid. The peroxyacid used in the present disclosure can be prepared using any method known in the art. When the peroxyacid is prepared from an acid and hydrogen peroxide, the resultant mixture contains both the peroxyacid and the corresponding acid that it is prepared from. For example, in embodiments that utilize peroxyacetic acid, the presence of the related acid (acetic acid) provides stability to the mixture, as the reaction is an equilibrium between the acid, hydrogen peroxide, and the peroxyacid and water, as follows:

H₂O₂+CH₃COOH [symbol] CH₃COO—OH+H₂O

Peracid salts, such as salts of the above listed peracids, can also be included as the peroxygen component of the solutions. Non-limiting examples of such salts include permanganates, perborates, perchlorates, peracetates, percarbonates, persulphates, and the like. The salts can be used alone or in combination with each other or other peroxygen compounds to form the peroxygen component of the disclosure.

In another embodiment, the peroxygen component of the disclosure can include a peroxide compound. While hydrogen peroxide is considered to be a desirable peroxide for use in accordance with embodiments of the present disclosure, other peroxides can also be used, such as metal peroxides and peroxyhydrates. The metal peroxides that can be used include, but are not limited to, sodium peroxide, magnesium peroxide, calcium peroxide, barium peroxide, and/or strontium peroxide. Other salts (for example sodium percarbonate) have hydrogen peroxide associated therewith much like waters of hydration, and these could also be considered to be a source of hydrogen peroxide, thereby producing hydrogen peroxide in situ. As mentioned above, the peroxides can be used alone or in combination with other peroxygen compounds to form the peroxygen component of the present disclosure.

Emollients, humectants, moisturizers, surfactants, skin nutrients, skin conditioners, skin protectants, herbs, and other skin health additives can also be used, such as carotenoids (e.g., astaxanthin), coconut oil as a moisturizer, and/or sorbitol or glycerol as an emollient as well as an alcohol. Likewise, a variety of humectants including those generally known in the art can be used. In one embodiment, the humectant can be a natural grain extract such as from wheat, oats, flax seed, and combinations thereof. Such natural extracts can be advantageous as they generally are less irritable to the skin. In one embodiment, the grain extract is gluten free. In another embodiment, the grain extract is a flax seed extract. Furthermore, examples of surfactants that can be used include, but are not limited to, liquid vegetable oil soap, sulfonated castor oil, sodium lauryl sulfate or any other liquid surfactant known in the art. Examples of skin protectants that can be used include, but are not limited to sunscreens, vitamin E, vitamin E derivatives, aloe vera gel, and combinations thereof.

The administration of the therapeutic aqueous composition can be done in any acceptable manner known in the medical and pharmaceutical arts. Specific non-limiting examples of topical administration include the use of fluids, aerosols, sprays, mists, lotions, creams, ointments, gels, gums, lozenges, suppository, drops, washes, dispensing bottles, squeeze tubes, pre-soaked fabric (cotton rounds, wipes, etc.), automatic mixing and/or dispensing devices, sprayers, bandages, transdermal patches or plasters, etc. Submersion of infected skin tissue is also an acceptable means of topical administration as well. The mode of administration can be dependent on the type and/or severity of the skin disease being treated and the formulated potency of the therapeutic aqueous composition. However, typically, it can be desirable to topically apply the aqueous composition to the areas where the infection is present or may shortly become present.

As stated above, the present disclosure is related to compositions, systems, and methods of treating (including prophylactically treating) skin disease. The amounts of the therapeutic aqueous compositions which can be administered using the methods of the present disclosure can vary depending on the type and location of the targeted infection, the mode of administration, and the potency or concentration of the aqueous composition administered. For example, when administered topically using a spray or submersion administration mode for topical local effect, the amount of aqueous composition may not be as important as the concentration of the aqueous composition and the frequency of administration may be more significant. In one embodiment, the administration can occur one or more times daily for a period of 1 day to 180 days. In another embodiment, the administration can occur one or more times daily for a period of 1 to 7 days. In another embodiment, the administration can occur one or more times for a period of 4 hours to 24 hours.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

### Compositions Usable for Treatment of Skin Disease or Infection

An aqueous composition is prepared which includes 0.1 wt % hydrogen peroxide, 4 wt % glycerol, 600 ppm of silver-zinc alloy, 7 wt % ethyl alcohol, 0.3 wt % salicylic acid, and the balance water. Optionally, additional skin health additives, such as emollients, carotenoids, skin nutrients, skin conditioners, skin protectants, and/or the like, can be substituted for a small portion of the water, e.g., up to 20 wt %. In one specific preparation scheme, the composition can be prepared by bringing two-parts (Part A and Part B) together prior to use and can be applied to the acne while reaction between Part A and Part B is occurring. For example, the silver-zinc alloy can be kept in Part A and the hydrogen peroxide can be kept in Part B. The other ingredients can typically be kept in either Part A and/or Part B.

### Example 2

### Compositions Usable for Treatment of Skin Disease or Infection

An aqueous composition is prepared which includes 0.05 wt % hydrogen peroxide acid, 8 wt % ethanol, 150 ppm by weight of colloidal silver, 0.5 wt % benzoyl peroxide, 3 wt % of a thickening agent (to form a non-runny gel or lotion), and the balance water. Optionally, additional skin health additives, such as emollients, carotenoids, skin nutrients, skin conditioners, skin protectants, and/or the like, can be substituted for a small portion of the water, e.g., up to 20 wt %. In one specific preparation scheme, the composition can be prepared by bringing two-parts (Part A and Part B) together prior to use and can be applied to the skin disease while reaction between Part A and Part B is occurring. For example, the silver-zinc alloy can be kept in Part A and the hydrogen peroxide can be kept in Part B. The other ingredients can typically be kept in either Part A and/or Part B.

### Example 3

### Compositions Usable for Treatment of Skin Disease or Infection

An aqueous composition is prepared which includes 10 wt % hydrogen peroxide, 2 wt % glycerol, 2 wt % sorbitol, 10,000 ppm of colloidal silver, 5,000 ppm colloidal zinc, 5 wt % ethyl alcohol, 1.5 wt % salicylic acid, and the balance water. Optionally, additional skin health additives, such as emollients, carotenoids, skin nutrients, skin conditioners, skin protectants, and/or the like, can be substituted for a small portion of the water, e.g., up to 20 wt %. In one specific preparation scheme, the composition can be prepared by bringing two-parts (Part A and Part B) together prior to use and can be applied to the skin disease while reaction between Part A and Part B is occurring. For example, the silver-zinc alloy can be kept in Part A and the hydrogen peroxide can be kept in Part B. The other ingredients can typically be kept in either Part A and/or Part B.

### Example 4

### Composition Usable for Treatment of Skin Disease or Infection

An aqueous composition suitable for treating skin disease is prepared which includes peroxide, alcohol, colloidal transition metal, a drug, water, and other ingredients, as set forth in Table 1 below:

TABLE 1

| | | | QUANTITY ON | |
|---|---|---|---|---|
| | | | ROUND AFTER | |
| | QUANTITY | | SPRAYING PART B | |
| | PER INDI- | | ONTO PRE-SOAKED | |
| 2-PART FORMULA | VIDUAL PART | | ROUND | |
| Part A - Soaked On Cotton | 115 | ml | 69.7 | wt % |
| Rounds (30 count 2.25 inch | | | | |
| Cotton Round Swisspers ®) | | | | |
| Silver Colloids | 300 | ppm (wt) | 209.09 | ppm (wt) |
| Zinc Colloids | 100 | ppm (wt) | 69.70 | ppm (wt) |
| Glycerol | 5 | wt % | 3.48 | wt % |
| Denatured Ethyl Alcohol | 10 | wt % | 6.97 | wt % |
| Part B - Spray Activator | 50 | ml | 30.3 | wt % |
| Hydrogen Peroxide (dilute | 5 | wt % | 1.52 | wt % |
| from concentrate) | | | | |
| Coconut Oil | 4 | wt % | 1.21 | wt % |
| Salicylic Acid | 1 | wt % | 0.30 | wt % |
| Skin Health Additives¹ | 0.5 | wt % | 0.15 | wt % |

¹Exemplary Skin Health Additives that can be used include benzoic peroxide, carotenoids (e.g., astaxanthin), skin nutrients, skin conditioners, skin protectants, and/or emollients.
Water is present in Part A and Part B up to 100 wt %.

### Example 5

### Composition Usable for Treatment of Skin Disease or Infection

An aqueous composition suitable for treating skin disease is prepared which includes peroxide, alcohol, colloidal transition metal, a drug, water, and other ingredients, as set forth in Table 2 below:

TABLE 2

| | | | QUANTITY ON | |
|---|---|---|---|---|
| | | | ROUND AFTER | |
| | QUANTITY | | SPRAYING PART B | |
| | PER INDI- | | ONTO PRE-SOAKED | |
| 2-PART FORMULA | VIDUAL PART | | ROUND | |
| Part A - Soaked On Cotton | 115 | ml | 69.7 | wt % |
| Rounds (30 count 2.25 inch | | | | |
| Cotton Round Swisspers ®) | | | | |
| Silver Colloids | 400 | ppm (wt) | 278.79 | ppm (wt) |
| Glycerol | 5 | wt % | 3.48 | wt % |
| Denatured Ethyl Alcohol | 10 | wt % | 6.97 | wt % |
| Part B - Spray Activator | 50 | ml | 30.3 | wt % |
| Hydrogen Peroxide (dilute | 3 | wt % | 0.91 | wt % |
| from concentrate) | | | | |
| Coconut Oil | 4 | wt % | 1.21 | wt % |
| Benzoyl Peroxide | 3 | wt % | 0.90 | wt % |
| Skin Health Additives¹ | 0.5 | wt % | 0.15 | wt % |

¹Exemplary Skin Health Additives that can be used include benzoic peroxide, carotenoids (e.g., astaxanthin), skin nutrients, skin conditioners, skin protectants, and/or emollients.
Water is present in Part A and Part B up to 100 wt %.

### Example 6

### Composition Usable for Treatment of Skin Disease or Infection

An aqueous composition suitable for treating skin disease is prepared which includes peroxide, alcohol, colloidal transition metal, a drug, water, and other ingredients, as set forth in Table 3 below:

TABLE 3

| | | | QUANTITY ON | |
|---|---|---|---|---|
| | | | ROUND AFTER | |
| | QUANTITY | | SPRAYING PART B | |
| | PER INDI- | | ONTO PRE-SOAKED | |
| 2-PART FORMULA | VIDUAL PART | | ROUND | |
| Part A - Soaked On Cotton | 85 | mL | 61.3 | wt % |
| Rounds (30 count 2.25 inch | | | | |
| Cotton Round Swisspers ®) | | | | |
| Salicylic Acid | 2 | wt % | 1.23 | wt % |
| Glycerol | 3.5 | wt % | 2.15 | wt % |
| Hydrogen Peroxide (dilute | 5 | wt % | 3.06 | wt % |
| from concentrate) | | | | |
| Ethyl Alcohol | 11 | wt % | 6.74 | wt % |
| Coconut Oil | 6 | wt % | 3.68 | wt % |
| Skin Health Additives¹ | 0.1 | wt % | 0.06 | wt % |
| Part B - Spray Activator | 60 | mL | 38.7 | wt % |

| | | | |
|---|---|---|---|
| Silver Colloids (449 ppm by | 80 wt % (metal | 139.05 | wt % |
| weight) | content) | | |
| Zinc Colloids (1100 ppm by | 20 wt % (metal | 85.16 | wt % |
| weight) | content) | | |

¹Exemplary Skin Health Additives that can be used include benzoic peroxide, carotenoids (e.g., astaxanthin), skin nutrients, skin conditioners, skin protectants, and/or emollients.
Water is present in Part A and Part B up to 100 wt %.

### Example 7

### Composition Usable for Treatment of Skin Disease or Infection

An aqueous composition suitable for treating skin disease is prepared which includes peroxide, alcohol, colloidal transition metal, a drug, water, and other ingredients, as set forth in Table 4 below:

TABLE 4

| | | | QUANTITY ON | |
|---|---|---|---|---|
| | | | ROUND AFTER | |
| | QUANTITY | | SPRAYING PART B | |
| | PER INDI- | | ONTO PRE-SOAKED | |
| 2-PART FORMULA | VIDUAL PART | | ROUND | |
| Part A - Soaked On Cotton | 85 | mL | 61.3 | wt % |
| Rounds (30 count 2.25 inch | | | | |
| Cotton Round Swisspers ®) | | | | |
| Glycerol | 5.5 | wt % | 3.38 | wt % |
| Hydrogen Peroxide (dilute | 5 | wt % | 3.06 | wt % |
| from concentrate) | | | | |
| Ethyl Alcohol | 11 | wt % | 6.74 | wt % |
| Coconut Oil | 6 | wt % | 3.68 | wt % |
| Skin Health Additives¹ | 0.1 | wt % | 0.06 | wt % |
| Part B - Spray Activator | 60 | mL | 38.7 | wt % |

| | | | |
|---|---|---|---|
| Silver Colloids (449 ppm by | 80 wt % (metal | 139.05 | wt % |
| weight) | content) | | |
| Zinc Colloids (1100 ppm by | 20 wt % (metal | 85.16 | wt % |
| weight) | content) | | |

¹Exemplary Skin Health Additives that can be used include benzoic peroxide, carotenoids (e.g., astaxanthin), skin nutrients, skin conditioners, skin protectants, and/or emollients.
Drug can be added to Part A and/or Part B at any appropriate amount.
Water is present in Part A and Part B up to 100 wt %.

### Example 8

### Composition Usable for Treatment of Skin Disease or Infection

An aqueous composition suitable for treating skin disease is prepared which includes peroxide, alcohol, colloidal transition metal, a drug, water, and other ingredients, as set forth in Table 5 below:

TABLE 5

| | | | QUANTITY ON | |
|---|---|---|---|---|
| | | | ROUND AFTER | |
| | QUANTITY | | SPRAYING PART B | |
| | PER INDI- | | ONTO PRE-SOAKED | |
| 2-PART FORMULA | VIDUAL PART | | PART A ROUND | |
| Part A - Soaked On Cotton | 100 | mL | 62.5 | wt % |
| Rounds (30 count 2.25 inch | | | | |
| Cotton Round Swisspers ®) | | | | |
| Glycerol | 5 | wt % | 3.13 | wt % |
| Hydrogen Peroxide (35% | 1 | wt % | 0.63 | wt % |
| concentrate) | | | | |
| Ethyl Alcohol | 15 | wt % | 9.38 | wt % |
| Acetylsalicylic Acid | 3 | wt % | 1.88 | wt % |
| Part B - Spray Activator | 60 | mL | 37.5 | wt % |
| AgSol (450 ppm colloidal | 97.5 | wt % | 164.5 ppm by weight | |
| silver by weight) | | | | |
| Herbs (background products) | 2.5 | wt % | 0.93 | wt % |

Water is present in Part A up to 100 wt %, whereas AgSol already includes water.
Dissolve Acetylsalicylic acid in solution at about 180° F.
10 fine mist sprays of Spray Activator per Cotton Round.
Herbs are shown as being used as background product, but these, or even the Skin Health Additives described previously can optionally be used.

### Example 9

### Treatment of Acne

Three separate studies were conducted using a formulation similar to those described in the examples, as set forth below:
a) A subject used a two-part composition daily by admixing the two-parts and immediately applying the resultant composition daily to the skin for a period of 2 weeks. The subject experienced a 95% reduction in my acne.
b) A subject that suffered from acne for over 10 years that had tried many products to limited success applied a two-part composition (immediately after admixing) to the face twice per day for 2 weeks. Most of the acne was gone at the end of the 2 week period.
c) A subject used a two-part composition daily by admixing the two-parts and immediately applying the resultant composition daily to the skin for a period of 1 month. The subject has been clear since the treatment ended.

### Example 10

### Treatment of Warts

Two separate studies were conducted using a formulation similar to that described in the examples, as set forth below:
a) A subject had a wart on the side of the cheek for over 3 years. After admixing and applying a two-part composition to the wart a few times a day for 4 days, the wart fell off.
b) A subject with many warts on the back began applying a two-part product (after admixing) to the warts daily. Within 2 weeks, the warts were nearly gone.

### Example 11

### Treatment of Fungal Infections

A study was conducted using a formulation similar to that described in the examples, as follows. A subject had an ugly fungal infection (dry, scaly patches) in many locations on the face. Within 3 days of applying the product (two-part composition admixed prior to use), the fungal patches were mostly clear. The subject continued using the product over a few weeks and the fungus ultimately cleared completely.

### Example 12

### Treatment of Bacterial Infection

A study was conducted using a formulation similar to that described in the examples, as follows. A subject reported that as child, the skin on the hands started to crack and peel. Doctors prescribed the use of plastic gloves with Nieva cream, which did not work, and often, became worse. The subject is lactose and glucose intolerant which causes leaky gut syndrome. After beginning to use the product for a period of 1 week, the subject's hands looked and felt significantly better.

### Example 13

### Treatment of Acne

Several separate subjects with acne and/or other skin conditions were treated specifically with the Formulation described in Example 8. Each subject used the product twice per day, and the results were as follows:
a) A female subject with mild to moderate acne did not feel comfortable without foundation makeup. After using the product consistently twice a day, bumps and dryness were alleviated, and the subject felt comfortable going out in public without foundation makeup.
b) A male subject with acne reported continued clear and rejuvenated skin after three months of use.
c) A female subject with moderate to severe acne reported clear skin after only one month of use. The subject also reported soft and smooth skin.
d) A male subject with acne and very sensitive skin (not able to use many acne products because it burns the skin surface) reported that with twice daily use, the skin was almost clear in two weeks. No burning of the skin surface was reported.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A composition suitable for treating skin disease, comprising:
water,
from 0.5 wt % to 25 wt % glycerol, sorbitol, or mixtures thereof;
from 1 wt % to 30 wt % of a C₁ to C₂₄ alcohol;
from 0.0001 wt % to 10.0 wt % of a peroxygen;
from 0.0001 ppm to 50,000 ppm by weight of a transition metal or alloy thereof; and
from 0.1 wt % to 8 wt % of a drug suitable for treating the skin disease.

2. The composition of claim 1, wherein the drug includes a member selected from the group consisting of benzoyl peroxide, salicylic acid, acetylsalicylic acid, sulfur, resorcinol, resorcinol monoacetate, amorolfine, butenafine, naftifine, terbinafine, fluconazole, itraconazole, ketoconazole, posaconazole, ravuconazole, voriconazole, clotrimazole, butoconazole, econazole, miconazole, oxiconazole, sulconazole, terconazole, tioconazole, caspofungin, micafungin, anidulafingin, amphotericin B, AmB, nystatin, pimaricin, griseofulvin, ciclopirox olamine, haloprogin, tolnaftate, undecylenate, acyclovir, penciclovir, famciclovir, valacyclovir, trifluridine, idoxuridine, cidofovir, gancyclovir, podofilox, podophyllotoxin, ribavirin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, rimantadine, zanamivir, erythromycin, clindamycin, tetracycline, bacitracin, neomycin, mupirocin, polymyxin B, quinolones, and imiquimod.

3. The composition of claim 1, wherein the drug includes benzoyl peroxide.

4. The composition of claim 1, wherein the drug includes salicylic acid.

5. The composition of claim 4, wherein the drug is acetylsalicylic acid.

6. The composition of claim 1, wherein the drug includes sulfur.

7. The composition of claim 1, wherein the drug includes resorcinol or resorcinol monoacetate, and optionally, further includes sulfur.

8. The composition of claim 1, wherein the transition metal or alloy thereof is selected from the group consisting of ruthenium, rhodium, osmium, iridium, palladium, platinum, copper, gold, silver, manganese, zinc, alloys thereof, and mixtures thereof.

9. The composition of claim 1, wherein the transition metal or alloy thereof is a colloidal transition metal or alloy thereof.

10. The composition of claim 9, wherein the colloidal transition metal or alloy thereof includes colloidal silver.

11. The composition of claim 9, wherein the colloidal transition metal or alloy thereof includes colloidal zinc.

12. The composition of claim 9, wherein the colloidal transition metal or alloy thereof includes a mixture or alloy of colloidal zinc and colloidal silver.

13. The composition of claim 9, wherein the colloidal transition metal or alloy thereof has an average particle size of from 0.030 μm to 0.5 μm.

14. The composition of claim 1, wherein the transition metal or alloy thereof is an ionic transition metal.

15. The composition of claim 1, wherein the transition metal or alloy thereof is present at from 0.0001 ppm to 1,500 ppm by weight.

16. The composition of claim 1, wherein the peroxygen is a peracid.

17. The composition of claim 16, wherein the peracid is selected from the group consisting of peroxyformic acid, peroxyacetic acid, peroxyoxalic acid, peroxypropanoic acid, perlactic acid, peroxybutanoic acid, peroxypentanoic acid, peroxyhexanoic acid, peroxyadipic acid, peroxycitric, peroxybenzoic acid, and mixtures thereof.

18. The composition of claim 1, wherein the peroxygen is a peroxide.

19. The composition of claim 1, wherein the peroxygen includes a peracid and a peroxide.

20. The composition of claim 1, wherein the peroxygen is present at from 0.05 wt % to 5.0 wt %.

21. The composition of claim 1, wherein the peroxygen is present at from 0.1 wt % to 1.5 wt %.

22. The composition of claim 1, wherein the C₁ to C₂₄ alcohol includes a member selected from the group consisting of methanol, ethanol, a propanol, a butanol, a pentanol, and mixtures thereof.

23. The composition of claim 1, formulated as an ointment, cream, mouth rinse, gel, lozenge, gum, wipe, dermal patch, foam, powder, aerosol, or bandage dressings.

24. The composition of claim 1, further comprising at least one skin health additive selected from the group consisting of herbs, emollients, carotenoids, skin nutrients, skin conditioners, and skin protectants.

25. The composition of claim 1, in the form of a reacting formulation, wherein at least two components of the composition are not in equilibrium at the time of application.

26. The composition of claim 1, comprising:
from 0.5 wt % to 10 wt % glycerol, sorbitol, or mixtures thereof;
from 1 wt % to 15 wt % C₁ to C₅ aliphatic alcohol;
from 0.0001 wt % to 10.0 wt % hydrogen peroxide;
from 0.0001 ppm to 10,000 ppm by weight colloidal silver; and
from 0.5 wt % to 5 wt % of the drug.

27. The composition of claim 26, wherein the drug is salicylic acid or acetylsalicylic acid.

28. A method of treating a skin disease, comprising applying the composition of claim 1 directly to a skin site afflicted with the skin disease.

29. A method of treating a skin disease, comprising applying the composition of claim 26 directly to a skin site afflicted with the skin disease.

30. A method of treating a skin disease, comprising applying the composition of claim 27 directly to a skin site afflicted with the skin disease.

31. A system suitable for treating skin disease, comprising:
a first container containing Part A of a two-part solution, Part A including a transition metal or alloy thereof; and
a second container containing Part B of the two-part solution, Part B including a peroxygen, an alcohol, and a drug; and
wherein upon combining Part A and Part B, a reacting formulation is formed that is effective for treating the skin disease.

32. The system of claim 31, wherein the drug includes a member selected from the group consisting of benzoyl peroxide, salicylic acid, acetylsalicylic acid, sulfur, resorcinol, resorcinol monoacetate, amorolfine, butenafine, naftifine, terbinafine, fluconazole, itraconazole, ketoconazole, posaconazole, ravuconazole, voriconazole, clotrimazole, butoconazole, econazole, miconazole, oxiconazole, sulconazole, terconazole, tioconazole, caspofungin, micafungin, anidulafingin, amphotericin B, AmB, nystatin, pimaricin, griseofulvin, ciclopirox olamine, haloprogin, tolnaftate, undecylenate, acyclovir, penciclovir, famciclovir, valacyclovir, trifluridine, idoxuridine, cidofovir, gancyclovir, podofilox, podophyllotoxin, ribavirin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, rimantadine, zanamivir, erythromycin, clindamycin, tetracycline, bacitracin, neomycin, mupirocin, polymyxin B, quinolones, and imiquimod.

33. The system of claim 31, wherein the drug includes benzoyl peroxide.

34. The system of claim 31, wherein the drug includes salicylic acid.

35. The system of claim 34, wherein the drug is acetylsalicylic acid.

36. The system of claim 31, wherein the drug includes sulfur.

37. The system of claim 31, wherein the drug includes resorcinol or resorcinol monoacetate, and optionally further includes sulfur.

38. The system of claim 31, wherein the transition metal or alloy thereof is selected from the group consisting of ruthenium, rhodium, osmium, iridium, palladium, platinum, copper, gold, silver, manganese, zinc, alloys thereof, and mixtures thereof.

39. The system of claim 31, wherein the transition metal or alloy thereof is a colloidal transition metal or alloy thereof.

40. The system of claim 39, wherein the colloidal transition metal or alloy thereof includes colloidal silver.

41. The system of claim 39, wherein the colloidal transition metal or alloy thereof includes colloidal zinc.

42. The system of claim 39, wherein the colloidal transition metal or alloy thereof includes a mixture or alloy of colloidal zinc and colloidal silver.

43. The system of claim 39, wherein the colloidal transition metal or alloy thereof has an average particle size of from 0.030 μm to 0.5 μm.

44. The system of claim 31, wherein the transition metal or alloy thereof is an ionic transition metal.

45. The system of claim 31, wherein the transition metal or alloy thereof is present in the reacting formulation at from 0.0001 ppm to 50,000 ppm by weight.

46. The system of claim 31, wherein the transition metal or alloy thereof is present in the reacting formulation at from 0.0001 ppm to 1,500 ppm by weight.

47. The system of claim 31, wherein the peroxygen is a peracid.

48. The system of claim 47, wherein the peracid is selected from the group consisting of peroxyformic acid, peroxyacetic acid, peroxyoxalic acid, peroxypropanoic acid, perlactic acid, peroxybutanoic acid, peroxypentanoic acid, peroxyhexanoic acid, peroxyadipic acid, peroxycitric, peroxybenzoic acid, and mixtures thereof.

49. The system of claim 31, wherein the peroxygen is a peroxide.

50. The system of claim 31, wherein the peroxygen includes a peracid and a peroxide.

51. The system of claim 31, wherein the peroxygen is present in the reacting formulation at from 0.0001 wt % to 10.0 wt % of a peroxygen.

52. The system of claim 31, wherein the peroxygen is present in the reacting formulation at from 0.05 wt % to 5.0 wt %.

53. The system of claim 31, wherein the peroxygen is present in the reacting formulation at from 0.1 wt % to 1.5 wt %.

54. The system of claim 31, wherein the alcohol includes a member selected from the group consisting of methanol, ethanol, propanols, butanols, pentanols, and mixtures thereof.

55. The system of claim 31, wherein the alcohol comprises glycerol.

56. The system of claim 31, wherein the alcohol comprises a combination of glycerol and a member selected from the group consisting of methanol, ethanol, propanols, butanols, pentanols, and mixtures thereof.

57. The system of claim 31, further comprising at least one skin health additive present in Part A or Part B.

58. The system of claim 57, wherein the skin health additive is selected from the group consisting of herbs, emollients, carotenoids, skin nutrients, skin conditioners, and skin protectants.

59. The system of claim 31, wherein the second container contains a plurality of skin wipes pre-soaked with Part B, and the first container is a dispenser adapted to dispense Part A onto a skin wipe to form a reacting formulation that is effective for treating the skin disease.

60. The system of claim 31, wherein the reacting formulation comprises:
water;
from 0.5 wt % to 25 wt % glycerol, sorbitol, or combinations thereof;
from 1 wt % to 30 wt % of a C₁ to C₅ aliphatic alcohol;
from 0.0001 wt % to 10.0 wt % of a peroxide;
from 0.0001 ppm to 50,000 ppm by weight of a transition metal or alloy thereof; and
from 0.1 wt % to 8 wt % of a drug suitable for treating the skin disease.

61. A method of treating a skin disease, comprising:
obtaining the system of claim 31;
combining Part A and Part B to form a reacting formulation; and
applying the reacting formulation to a skin site afflicted with the skin disease.

62. A composition suitable for treating skin disease, comprising:
water;
an alcohol;
a peroxide;
a transition metal or alloy thereof; and
acetylsalicylic acid.

63. The composition of claim 62, wherein the transition metal or alloy thereof is selected from the group consisting of ruthenium, rhodium, osmium, iridium, palladium, platinum, copper, gold, silver, manganese, zinc, alloys thereof, and mixtures thereof.

64. The composition of claim 62, wherein the transition metal or alloy thereof is a colloidal transition metal or alloy thereof.

65. The composition of claim 64, wherein the colloidal transition metal or alloy thereof includes colloidal silver.

66. The composition of claim 62, wherein the transition metal or alloy thereof is an ionic transition metal.

67. The composition of claim 62, wherein the transition metal or alloy thereof is present at from 0.0001 ppm to 1,500 ppm by weight.

68. The composition of claim 62, wherein the peroxygen is present at from 0.05 wt % to 5.0 wt %.

69. The composition of claim 62, wherein the alcohol includes a member selected from the group consisting of methanol, ethanol, a propanol, a butanol, a pentanol, and mixtures thereof.

70. The composition of claim 62, wherein the alcohol includes glycerol.

71. The composition of claim 62, formulated as an ointment, cream, mouth rinse, gel, lozenge, gum, wipe, dermal patch, foam, powder, aerosol, or bandage dressings.

72. The composition of claim 62, further comprising at least one skin health additive selected from the group consisting of herbs, emollients, carotenoids, skin nutrients, skin conditioners, and skin protectants.

73. The composition of claim 62, in the form of a reacting formulation, wherein at least two components of the composition are not in equilibrium at the time of application.

74. The composition of claim 62, comprising:
from 0.5 wt % to 10 wt % glycerol;
from 1 wt % to 15 wt % ethyl alcohol;
from 0.0001 wt % to 10.0 wt % hydrogen peroxide;
from 0.0001 ppm to 10,000 ppm by weight colloidal silver; and
from 0.5 wt % to 5 wt % of the acetylsalicylic acid.

75. A method of treating a skin disease, comprising applying the composition of claim 62 directly to a skin site afflicted with the skin disease.

76. A system suitable for treating skin disease, comprising:
a first container containing Part A of a two-part solution, Part A including a transition metal or alloy thereof;
a second container containing Part B of the two-part solution, Part B including a peroxygen; and
acetylsalicylic acid present in at least one of Part A, Part B, or a third formulation,
wherein upon combining Part A and Part B, a reacting formulation is formed that, in combination with the acetylsalicylic acid, is effective for treating the skin disease.

77. The system of claim 76, wherein the transition metal or alloy thereof is selected from the group consisting of ruthenium, rhodium, osmium, iridium, palladium, platinum, copper, gold, silver, manganese, zinc, alloys thereof, and mixtures thereof.

78. The system of claim 76, wherein the transition metal or alloy thereof is a colloidal transition metal or alloy thereof.

79. The system of claim 78, wherein the colloidal transition metal or alloy thereof includes colloidal silver.

80. The system of claim 76, wherein the transition metal or alloy thereof is an ionic transition metal.

81. The system of claim 76, wherein the transition metal or alloy thereof is present in the reacting formulation at from 0.0001 ppm to 50,000 ppm by weight.

82. The system of claim 76, wherein the transition metal or alloy thereof is present in the reacting formulation at from 0.0001 ppm to 1,500 ppm by weight.

83. The system of claim 76, wherein the peroxygen is a peracid.

84. The system of claim 83, wherein the peracid is selected from the group consisting of peroxyformic acid, peroxyacetic acid, peroxyoxalic acid, peroxypropanoic acid, perlactic acid, peroxybutanoic acid, peroxypentanoic acid, peroxyhexanoic acid, peroxyadipic acid, peroxycitric, peroxybenzoic acid, and mixtures thereof.

85. The system of claim 76, wherein the peroxygen is a peroxide.

86. The system of claim 76, wherein the peroxygen includes a peracid and a peroxide.

87. The system of claim 76, wherein the peroxygen is present in the reacting formulation at from 0.05 wt % to 5.0 wt %.

88. The system of claim 76, further comprising an alcohol in Part B.

89. The system of claim 88, wherein the alcohol includes a member selected from the group consisting of methanol, ethanol, propanols, butanols, pentanols, and mixtures thereof.

90. The system of claim 88, wherein the alcohol comprises glycerol.

91. The system of claim 76, further comprising at least one skin health additive present in Part A or Part B.

92. The system of claim 91, wherein the skin health additive is selected from the group consisting of herbs, emollients, carotenoids, skin nutrients, skin conditioners, and skin protectants.

93. The system of claim 76, wherein the second container contains a plurality of skin wipes pre-soaked with Part B, and the first container is a dispenser adapted to dispense Part A onto a skin wipe to form a reacting formulation that is effective for treating the skin disease.

94. The system of claim 76, wherein the reacting formulation comprises:
water;
from 0.5 wt % to 25 wt % glycerol, sorbitol, or combinations thereof;
from 1 wt % to 30 wt % of a C₁ to C₂₄ alcohol;
from 0.0001 wt % to 10.0 wt % of a peroxide;
from 0.0001 ppm to 50,000 ppm by weight of a transition metal or alloy thereof; and
from 0.1 wt % to 8 wt % of the acetylsalicylic acid.

95. A method of treating a skin disease, comprising:
obtaining the system of claim 76;
combining Part A and Part B to form a reacting formulation; and
applying the reacting formulation along with the acetylsalicylic acid to a skin site afflicted with the skin disease.

96. The method of claim 95, wherein the skin disease is an inflammatory infection.

97. The method of claim 96, wherein the inflammatory disease includes acne, aczema, dermatitis, poison ivy, psoriasis, pyoderma gangrenosum, rosacea, hives, or burns.

98. The method of claim 95, wherein the skin disease is a bacterial skin infection.

99. The method of claim 98, wherein the bacterial skin infection includes impetigo, folliculitis, furunculosis, carbunculosis, eethyma, erysipelas, cellulitis, or necrotizing fasciitis.

100. The method of claim 95, wherein the skin disease includes a fungal or yeast infection.

101. The method of claim 100, wherein the fungal or yeast infection includes dermatophytosis, candidiasis, tinea, athlete's foot, nail fungal infection, or diaper rash.

102. The method of claim 95, wherein the skin disease includes a viral infection.

103. The method of claim 102, wherein the viral infection includes herpes simplex, herpes zoster, cold sores, warts, or molluscum contagiosum.

104. The method of claim 95, wherein the skin disease includes an infection caused by small macro organism selected from mites, insects, and bugs.

105. The method of claim 95, wherein the transition metal or alloy thereof is a colloidal transition metal.

108. The method of claim 95, wherein Part B further comprises an alcohol.

109. The method of claim 95, wherein the peroxygen is a peroxide.

110. The method of claim 95, wherein the acetylsalycilic acid is present in Part B.
